(19) United States
(12) Patent Application Publication
(10) Pub. No.: US 2016/0367393 A1
(43) Pub. Date: Dec. 22, 2016

(54) NASAL IMPLANT FOR CORRECTION

(71) Applicant: Hyun seok YOU, Seoul (KR)

(72) Inventor: Hyun seok YOU, Seoul (KR)

(21) Appl. No.: 14/901,682

(22) PCT Filed: Jun. 17, 2014
(86) PCT No.: PCT/KR2014/005277

§ 371 Date: Dec. 28, 2015

(30) Foreign Application Priority Data
Jun. 28, 2013 (KR) 10-2013-0075797

(51) Int. Cl. A61F 5/08; A61F 2/18; A61F 2/00

(52) U.S. Cl. CPC A61F 5/08; A61F 2/0059; A61F 2/186

(57) ABSTRACT

An implant inserted into the nasal cavity for correcting the shape of the nose, and more particularly, an implant including a core material part having flexibility and an outer skin material part having more flexibility than the core material part. The implant may correct the bridge of the nose just with a simple structure which is entirely inserted into the nasal cavity without being substantially exposed outwards, and may also correct the shape of the nose as desired by adjusting size, flexibility or the like of the core material part and the outer skin material part. Therefore, the shape of the nose may be simply corrected by inserting the implant without any surgical procedure, and during use for a long period of time, the shape of the nasal cartilage changes, which may give a plastic surgery effect to some extent.

### CROSS REFERENCE TO RELATED APPLICATION

This application claims benefit under 35 U.S.C. 119(e), 120, 121, or 365(c), and is a National Stage entry from International Application No. PCT/KR2014/005277 filed Jun. 17, 2014, which claims priority to and the benefit of Korean Patent Application No. 10-2013-0075797 filed in the Korean Intellectual Property Office on Jun. 28, 2013, the entire contents of which are incorporated herein by reference.

### TECHNICAL FIELD

The present disclosure relates to an implant inserted into the nasal cavity for correcting the shape of the nose, and more particularly, to an implant including a core material part having flexibility and an outer skin material part having more flexibility than the core material part.

### BACKGROUND ART

The nose which is a part of respiratory organs and a sensory organ responsible for the sense of smell is anatomically composed of a nasal portion protruding from the face and a nasal cavity constituting the inside thereof. The shape of the nose is basically formed with the fibula, the cartilage, and a frontal projection which is a part of the upper jawbone, and also includes nasal muscle and skin additionally. The fibula is provided in pair, and the nasal muscle is provided based on a long rectangular thin bone to stand in line in contact with a midline and cover the nasal cavity from an upper front side. One pair of outer cartilages having a flat triangular shape extends at the lower edge of the fibula to form a part of the bottom front wall of the nose. The outer side of the fibula is coupled to the frontal projection of the upper jawbone, and at the outer cartilage, one pair of alar cartilages surrounds the rounded sides and nostrils of the nose at their fronts and becomes a basis of the end of the nose. The nasal septum dividing the nasal cavity into right and left parts based on the midline has a front portion which serves as a basis of the nasal septum cartilage having an asymmetric diamond shape. The front edge of the cartilage extends to the bridge of the nose and connects to the right and left outer cartilage.

Recently, as the interest on the appearance is increasing, cosmetic surgeries are rampant to change and supplement the appearance through surgical procedures for better self-confidence and better life. Particularly, the shape of the noise located at the center of the face gives a great influence on the impression of the face, and thus high expectations are being placed on the plastic surgery of the nose. Recently, a large and high nose like a westerner is preferred, the rhinoplasty tends to prevail.

When the rhinoplasty is applied, since the lower portion of the nose is anatomically composed of cartilage, it is preferred to use autologous cartilage such as ear cartilage or nasal septum cartilage, which has biocompatibility and suitable properties, but extracting the autologous cartilage requires significant costs and additional surgeries.

Therefore, a silicon implant is commonly used since the silicon implant is inexpensive, is harmless to the human body, allows minute carving suitable for a bond shape, and has no change in size and shape after surgery. However, the silicon has its specific hardness and thus does not give the feeling of cushion or elasticity like a skin. Therefore, the silicon implant has a limit in enhancing the satisfactory of a user and may cause side effects such as infection or pain due to the silicon after surgery or disfigure the entire face.

In addition, after surgery, external activities are restricted for a certain period, inconveniently, and the change of the appearance may be easily recognized by other persons. In addition, due to psychological anxiety on surgery failure or side effects, a person interested in surgery may not easily determine to have the surgery.

Accordingly, an implant or prosthetics capable of changing the shape of the nose without any surgical procedure is greatly interested. For example, Korean Patent Registration No. 0357454 discloses a nose plastic surgery machine, which includes an expandable connection rod for connecting a shaping cap and a support cap, and a spring for giving an elastic force to the shaping cap so that the shaping cap may press the bridge of sharply. However, the existing nose plastic surgery machine is inserted into the nose to continuously push a specific portion at the inside of the nose by means of an elastic force of an elastic member, and thus a pain occurs at the specific portion supported thereby if the machine is worn for a long time, thereby causing resistance to the wearer. In addition, the machine is easily released if the wearer blows or touches the nose, and the machine may give a scar to the nose due to an external impact. In addition, the nose plastic surgery machine has many components, which may increase a manufacture cost, and if any one of these components is lost or broken, the entire machine may not be used.

Therefore, there is demanded a prosthetics or implant, which may have a simple structure and minimize the inconvenience of a wearer.

### SUMMARY

In order to solve the problems of the related art, the inventors have studied and endeavored to develop an implant capable of correcting the shape of the nose with a simple structure, and finally completed the present disclosure by finding that the shape of the nose can be corrected if an implant includes a core material part having flexibility and an outer skin material part having more flexibility than the core material part so that the core material part inserted into the nasal cavity is located at the nasal septum and both ends of the outer skin material part apply force to the outer cartilage.

Therefore, an object of the present disclosure is to provide an implant which may correct the shape of the nose by including a core material part and an outer skin material part with different flexibilities.

In one aspect of the present disclosure, there is provided an implant inserted into the nasal cavity to correct the shape of the nose, comprising: a core material part having flexibility; and an outer skin material part having more flexibility than the core material part.

In addition, the outer skin material part may surround an entire surface of the core material part, and the implant may have a thickness kept constantly to have a plate shape.

In addition, a loop portion may be formed at a surface of the outer skin material part to be inserted into the nasal cavity.

In addition, the core material part may be inserted into the nasal cavity and located at the nasal septum, and both ends of the outer skin material part may be inserted into the nasal cavity and located at outer cartilage.

The implant of the present disclosure may correct the bridge of the nose just with a simple structure which is entirely inserted into the nasal cavity without being substantially exposed outwards, and may also correct the shape of the nose as desired by adjusting size, flexibility or the like of the core material part and the outer skin material part. Therefore, the shape of the nose may be simply corrected by inserting the implant without any surgical procedure, and during use for a long period of time, the shape of the nasal cartilage changes, which may give a plastic surgery effect to some extent.

In addition, the implant of the present disclosure may be easily stored and washed due to a very small size as a whole, thereby being always kept in a hygienic state.

Moreover, the implant of the present disclosure may enlarge the nasal cavity, and this it is expected that the implant may be used as a nose respiratory assisting tool for patients of sinus infection, sinus cold, snoring or the like.

### BRIEF DESCRIPTION OF DRAWINGS

FIG. 1 is a cross-sectional view showing an implant according to the first embodiment of the present disclosure.

FIG. 2 is a cross-sectional view showing that a force is applied to both ends of the implant according to the first embodiment of the present disclosure to be bent.

FIG. 3 is a cross-sectional view showing an implant according to the first embodiment of the present disclosure, whose whole thickness is changed.

FIG. 4 is a perspective view showing the implant according to the first embodiment of the present disclosure.

FIG. 5 is a perspective view showing an implant according to the second embodiment of the present disclosure.

FIG. 6 is a perspective view showing an implant according to the third embodiment of the present disclosure.

FIG. 7 shows an example where the implant according to the first embodiment of the present disclosure is inserted into the nasal cavity.

FIG. 8 shows a state where the implant according to the first embodiment of the present disclosure is inserted into the nasal cavity, observed from the below.

### DETAILED DESCRIPTION

Advantages, features and measures of the present disclosure will be understood from the embodiments described in detail with reference to the accompanying drawings. However, it should be understood that the present disclosure is not limited to the embodiments disclosed below but may be implemented in various ways. The embodiments are just for better understanding of the present disclosure to those skilled in the art, and the scope of the present disclosure is just defined based on the appended claims. Throughout the specification, like reference numerals denote like elements.

Meanwhile, thickness or size of some regions is exaggerated to clearly show each region.

Hereinafter, an implant for correcting the shape of the nose according to the present disclosure will be described in detail.

Implant for Correcting the Shape of the Nose

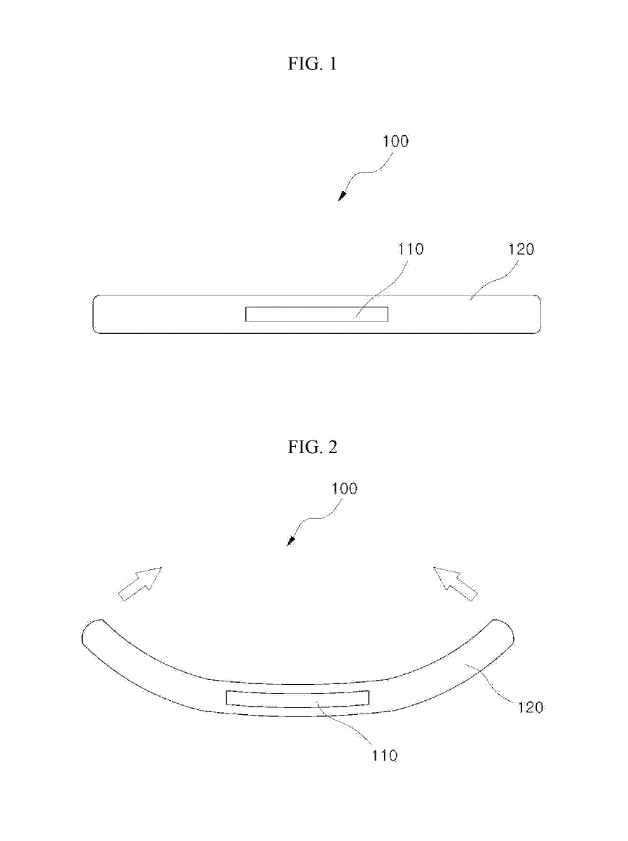

FIG. 1 is a cross-sectional view showing an implant 100 according to the first embodiment of the present disclosure, and FIG. 2 is a cross-sectional view showing that a force is applied to both ends of the implant 100 according to the first embodiment of the present disclosure in an arrow direction so that the implant 100 is bent.

The implant 100 of the present disclosure includes a core material part 110 having flexibility and an outer skin material part 120 having more flexibility than the core material part. At this time, the flexibility means a bending property when an article receives a rotating force from the outside, and the implant 100 of the present disclosure may be bent if a certain force is applied to both ends thereof. However, as shown in FIG. 2, a central portion of the implant 100 where the core material part 110 is included has less flexibility and thus not easily bent, but both end portions of the implant 100 where the core material part 110 is not included but only the outer skin material part 120 is provided have relatively greater flexibility and thus are more easily bent.

The outer skin material part 120 contacts the inner surface of the nasal cavity corresponding to the space in the nose when the implant 100 is inserted into the nasal cavity, and thus the outer skin material part 120 may be made of biocompatible polymer with flexibility, which may employ plastic such as polyethylene terephthalate (PET), polyvinyl chloride (PVC), polymethyl methacrylate (PMMA), polystyrene (PS), polypropylene (PP), polyurethane (PU) and polysulfone, rubber, silicon or the like.

The core material part 110 may employ any material with greater flexibility than the outer skin material part 120, without limitation, but may be made of plastic such as polyethylene terephthalate (PET), polyvinyl chloride (PVC), polymethyl methacrylate (PMMA), polystyrene (PS), polypropylene (PP), polyurethane (PU) and polysulfone, rubber, silicon or the like.

The outer skin material part 120 is formed to surround the surface of the core material part 110. A part of the core material part 110 may be exposed outwards, but it is more advantageous that the outer skin material part 120 surrounds the entire surface of the core material part 110 in order to ensure consistent appearance design and suitable flexibility and reduce feeling of irritation in use. However, the core material part 110 is located at a center portion of the implant 100 in a length direction, both ends of the implant 100 may have a symmetric shape composed of only the outer skin material part 120, but it is also possible that the core material part 110 is not located at the center in the length direction of the implant 100 but deviated in one side so that the end portions composed of only the outer skin material part 120 are asymmetric with different lengths. In addition, the core material part 110 may also be formed at one extreme end of the implant 100 so that an end portion composed of only the outer skin material part 120 is formed only at one side. Meanwhile, the length of the core material part 110 may be in the range of ½ to ⅕ of the entire length of the implant 100 so that the core material part 110 may be stably supported and also apply a suitable force to the nasal septum when being inserted into the nasal cavity.

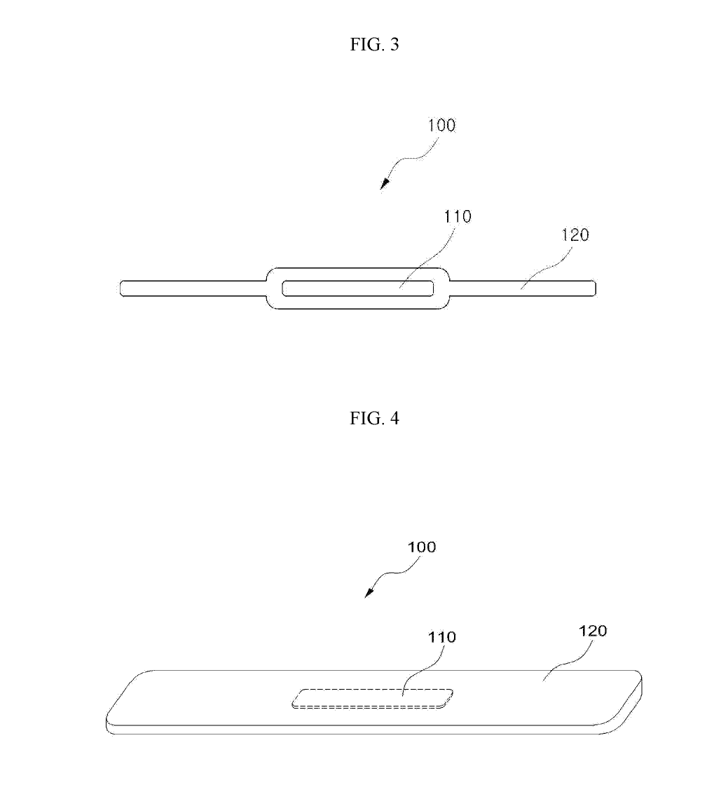

In addition, the implant 100 of the present disclosure may have a greater thickness at a portion where the core material part 110 is formed as shown in FIG. 3, but it is also possible that the implant 100 has a regular thickness as a whole to have a plate shape, which may advantageously eliminate feeling of irritation from the user.

FIG. 4 is a perspective view showing the implant 100 according to the first embodiment of the present disclosure.

As shown in FIG. 4, the implant 100 may have an approximately rectangular strip shape where a lateral size is larger than a longitudinal side, and each corner may be rounded in order to prevent the inner skin of the nasal cavity from being damaged. In addition, the corner may be separately coated to prevent the skin from being damaged.

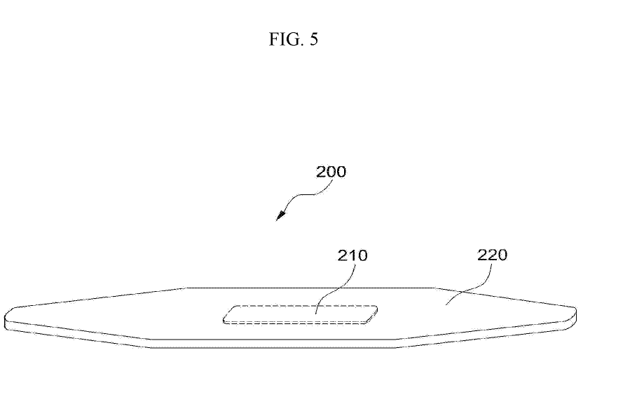

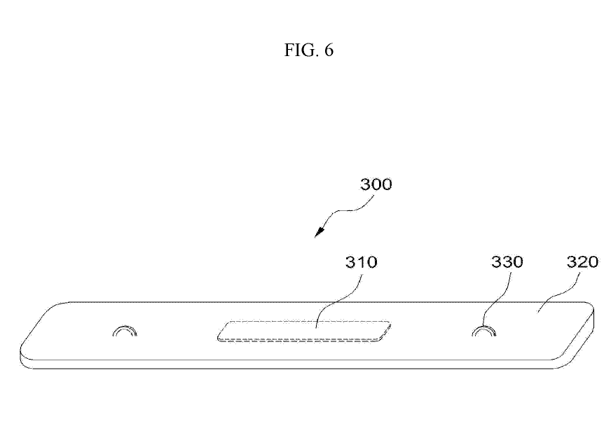

FIG. 5 is a perspective view showing an implant 200 according to the second embodiment of the present disclosure.

As shown in FIG. 5, the implant 200 may be formed so that both ends, namely the portions composed of only an outer skin material part 220 have gradually decreasing widths, and also the implant 200 may have an oval shape elongated in a lateral direction as a whole or be suitably modified into a design with good aesthetic sense.

FIG. 6 is a perspective view showing an implant 300 according to the third embodiment of the present disclosure.

As shown in FIG. 6, a loop portion 330 may be formed at the surface of the outer skin material part 320 for the insertion into the nasal cavity, and preferably, a pair of loop portions 330 may be formed at both ends of the implant 300. A wearer or operator who inserts the implant 300 into the nasal cavity may use a tool such as pincers at the loop portion 330 so that the implant may be easily inserted into or drawn from the nasal cavity.

Correction of the Shape of the Nose by the Insertion of the Implant

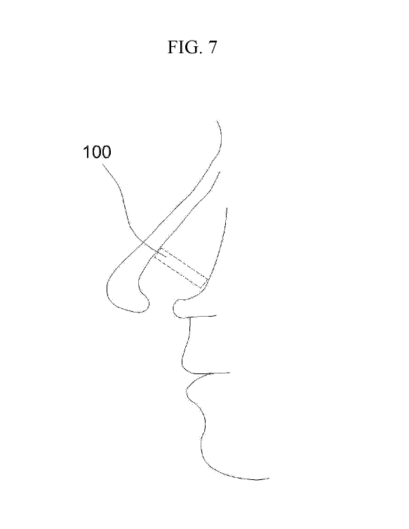

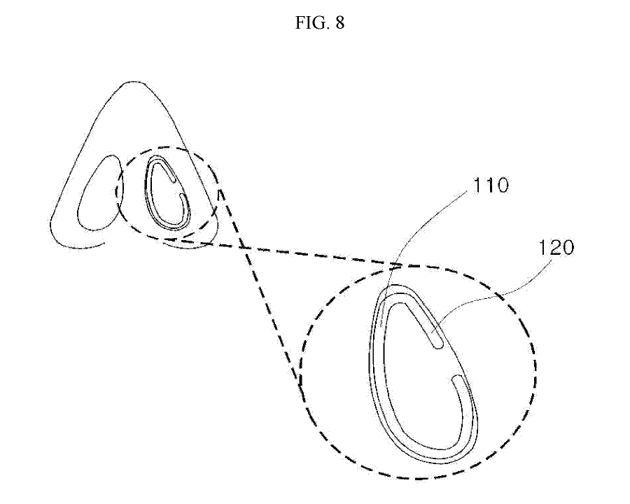

FIG. 7 shows an example where the implant according to the first embodiment of the present disclosure is inserted into the nasal cavity, and FIG. 8 shows an inner state of the nasal cavity, observed from a location below the nose (the right circle is an enlarged view of the left circle portion).

As shown in FIGS. 7 and 8, in a state where a force is applied to both ends of the implant 100 to be compressed, the implant 100 may be entirely inserted to correct the shape of the nose.

If the implant 100 is inserted into the nasal cavity, the core material part 110 is located at the nasal septum which corresponds to a wall serving as a border between the right and left nasal cavities, and both ends of the outer skin material part 120 are located at the outer cartilage. At this time, due to the elastic force of the outer skin material part 120 having flexibility, a force is applied in the inward direction of the nasal septum, and according to forces applied at right and left sides of the nasal septum, the bridge of the nose is naturally erected to make a sharp nose. In addition, the core material part 110 has relatively smaller flexibility than the outer skin material part 120 and thus may play a role of fixing the implant 100 to the nasal septum.

Meanwhile, according to the size of the nasal cavity of the user and the entire length of the implant 100, both ends of the outer skin material part 120 inserted into the nasal cavity may overlap each other or be spaced apart from each other. In a state where the implant 100 is inserted, the location of the implant 100 may be adjusted by applying a suitable force at the outside of the nose by using the finger of the user, and the intensity of force applied to the nasal septum may be adjusted by using the elastic force of the implant 100.

The implant 100 according to the present disclosure is entirely inserted into the nasal cavity and thus not shown from the outside, thereby protecting privacy of the user.

In particular, in case of the implant according to the third embodiment of the present disclosure as shown in FIG. 6, a tool such as pincers may be put into the loop portion 330, deformed into a C shape and inserted into the nasal cavity, so that the implant 300 may be easily settled at a suitable location in the nasal cavity. Meanwhile, the implant 300 inserted into the nasal cavity may be easily taken out of the nasal cavity by putting a tool such as pincers into the loop portion 330.

Even though the present disclosure has been described with reference to the embodiments depicted in the drawings, it should be understood that these embodiments are just for illustration, and various modifications and equivalents can be made therefrom by those skilled in the art. Therefore, the scope of the present disclosure should be defined based on the appended claims, and all technical features equivalent thereto should be defined as falling within the scope of the present disclosure.

What is claimed is:

1. An implant inserted into the nasal cavity to correct the shape of the nose, comprising:
a core material part having flexibility; and
an outer skin material part having more flexibility than the core material part.

2. The implant according to claim 1, wherein the outer skin material part surrounds an entire surface of the core material part.

3. The implant according to claim 1, wherein the implant has a thickness kept constantly to have a plate shape.

4. The implant according to claim 1, wherein a loop portion is formed at a surface of the outer skin material part to be inserted into the nasal cavity.

5. The implant according to claim 1, wherein the core material part is inserted into the nasal cavity and located at the nasal septum, and both ends of the outer skin material part is inserted into the nasal cavity and located at outer cartilage.
